SEQUENCE LISTING (41 sequences)

SEQ ID NO: 1 - DNA, 22 bp, Artificial Sequence
  oligonucleotide
cctcctactatccctaagagcg

SEQ ID NO: 2 - DNA, 39 bp, Artifical Sequence
  oligonucleotide
gggccaggatgatgccatccacaggaacctgaaattgac

SEQ ID NO: 3 - DNA, 20 bp, Artificial Sequence
  oligonucleotide
ccctaagagcgagtgaaagg

SEQ ID NO: 4 - DNA, 39 bp, Artificial Sequence
  oligonucleotide
gtcaatttcaggttcctgtggatggcatcatcctggccc

SEQ ID NO: 5 - DNA, 20 bp, Artificial Sequence
  oligonucleotide
agggagctgttagtgaaggc

SEQ ID NO: 6 - DNA, 20 bp, Artificial Sequence
  oligonucleotide
ggccgaaggatagaaagacc

SEQ ID NO: 7 - DNA, 31 bp, Artificial Sequence
  oligonucleotide
cggcggccgctcatcaagtccagggattagg

SEQ ID NO: 8 - DNA, 29 bp, Artificial Sequence
  oligonucleotide
gcgctagctcatcaagtccagggattagg

SEQ ID NO: 9 - DNA, 29 bp, Artificial Sequence
  oligonucleotide
gcgtcgactggttattcctacagcaacgg

SEQ ID NO: 10 - DNA, 28 bp, Artificial Sequence
  oligonucleotide
gcgtcgactgtgagatgcacacgtcagc

SEQ ID NO: 11 - DNA, 20 bp, Artificial Sequence
  oligonucleotide
ttagccatggttcagtcacg

SEQ ID NO: 12 - DNA, 21 bp, Artificial Sequence
  oligonucleotide
cacgactcctgtgaatcaagg

SEQ ID NO: 13 - DNA, 20 bp, Artificial Sequence
  oligonucleotide
aaggctgtagcttcagcgag

SEQ ID NO: 14 - DNA, 20 bp, Artificial Sequence
  oligonucleotide
tttctgctatggttaggcgc

SEQ ID NO: 15 - DNA, 20 bp, Artificial Sequence
  oligonucleotide
aaggatggtggagttgaacg

SEQ ID NO: 16 - DNA, 19 bp, Artificial Sequence
  oligonucleotide
aagcaggttggatcctacg

SEQ ID NO: 17 - DNA, 44 bp, Artificial Sequence
  oligonucleotide
cttccaacttttctggcggaattaagtgatgcacctctttataa

SEQ ID NO: 18 - DNA, 28 bp, Artificial Sequence
  oligonucleotide
gcgctagccaaacttggcaaatcagagc

SEQ ID NO: 19 - DNA, 44 bp, Artificial Sequence
  oligonucleotide
ttataaagaggtgcatcacttaattccgccagaaaagttggaag

SEQ ID NO: 20 - DNA, 28 bp, Artificial Sequence
  oligonucleotide
gcgctagccaaacttggcaaatcagagc

SEQ ID NO: 21 - DNA, 21 bp, Artificial Sequence
  oligonucleotide
cacgactcctgtgaatcaagg

SEQ ID NO: 22 - DNA, 28 bp, Artificial Sequence
  oligonucleotide
ggtcgactggttattcctacagcaacgg

SEQ ID NO: 23 - DNA, 27 bp, Artificial Sequence
  oligonucleotide
ggtcgactgaaccaaggccacactctc

SEQ ID NO: 24 - DNA, 20 bp, Artificial Sequence
  oligonucleotide
ctcaacaaaccctggacatc

SEQ ID NO: 25 - DNA, 20 bp, Artificial Sequence
  oligonucleotide
gaagtctgaggagagaagtg

SEQ ID NO: 26 - DNA, 20 bp, Artificial Sequence
  oligonucleotide
ttctaaccaaagccagcagg

SEQ ID NO: 27 - DNA, 20 bp, Artificial Sequence
  oligonucleotide
ggcagaggtcacttccaaag

SEQ ID NO: 28 - DNA, 21 bp, Artificial Sequence
  oligonucleotide
tcttattccaggtgcaagcag

SEQ ID NO: 29 - DNA, 24 bp, Artificial Sequence
  oligonucleotide
gggatgaagatattggagcaagac

SEQ ID NO: 30 - DNA, 19 bp, Artificial Sequence
  oligonucleotide
ctggtctgccatcataacg

SEQ ID NO: 31 - DNA, 20 bp, Artificial Sequence
  oligonucleotide
caggatcagcctaatctccg

SEQ ID NO: 32 - DNA, 37 bp, Artificial Sequence
  oligonucleotide
ctgaccgcttcctcctgaccgcttcctcgtgctttac

SEQ ID NO: 33 - DNA, 19 bp, Artificial Sequence
  oligonucleotide
cggaggacagagtaatcgg

SEQ ID NO: 34 - DNA, 17 bp, Artificial Sequence
  oligonucleotide
gaggacagagtaatcgg

SEQ ID NO: 35 - DNA, 40 bp, Artificial Sequence
  oligonucleotide
gctccctgttccgagttagggctccctgttccgagttagg

SEQ ID NO: 36 - DNA, 25 bp, Artificial Sequence
  oligonucleotide
agctgtgagctctttgtttttctgt

SEQ ID NO: 37 - DNA, 24 bp, Artificial Sequence
  oligonucleotide
tttggttagaaggctgtagcttca

SEQ ID NO: 38 - DNA, 22 bp, Artificial Sequence
  oligonucleotide
ggacgccatccacaggaaccca

SEQ ID NO: 39 - DNA, 23 bp, Artificial Sequence
  oligonucleotide
gctgaagctgtgagctctttgtt

SEQ ID NO: 40 - DNA, 23 bp, Artificial Sequence
  oligonucleotide
ggttggaggattggagctctaga

SEQ ID NO: 41 - DNA, 24 bp, Artificial Sequence
  oligonucleotide
tgccatccacaggaacccagacag

(19) United States
(12) Patent Application Publication
(10) Pub. No.: US 2009/0007283 A1
(43) Pub. Date: Jan. 1, 2009

(54) Transgenic Rodents Selectively Expressing Human B1 Bradykinin Receptor Protein

(76) Inventors: John W. Hess, Lansdale, PA (US); Robert J. Gould, Beverly, MS (US); Douglas J. Pettibone, Chalfont, PA (US); Thomas F. Vogt, Princeton, NJ (US); Richard Z. Chen, Scotch Plains, NJ (US)

Correspondence Address: MERCK AND CO., INC, P O BOX 2000, RAHWAY, NJ, 07065-0907, US

(21) Appl. No.: 10/573,926

(22) PCT Filed: Oct. 4, 2004
(86) PCT No.: PCT/US2004/032505

§ 371 Date: Mar. 30, 2006

Related U.S. Application Data
(60) Provisional application No. 60/509,505, filed on Oct. 8, 2003.

(51) Int. Cl. A01K 67/027

(52) U.S. Cl. 800/18; 800/13

(57) ABSTRACT

Non-human transgenic animals, such as transgenic mice, are generated which incorporate the a non-native form of the bradykinin B1 receptor gene against a null phenotype for the native form of the bradykinin B1 receptor. An exemplified portion of the invention disclosed a transgenic mouse wherein a targeting construct containing a transgene encoding the human B1 bradykinin receptor gene is inserted downstream of and operatively linked to the native mice bradykinin B1 promoter. This targeting construct also contains a fluxed neomycin resistance gene. The resulting transgenic animals are “humanized” for the bradykinin B1 receptor and are effectively on a null background for native, functional B1 receptor activity. These animals may be crossed with a Cre-deleter strain to generate transgenic offspring which absent of the floxed marker gene. The transgenic animals described herein provide for a model to The transgenic mice of the present invention provide for an animal model enabling the analysis of compounds that are selective for the human B1 bradykinin receptor, relative to the rodent (e.g., rat or mouse) B1 bradykinin receptor.

### CROSS-REFERENCE TO RELATED APPLICATIONS

This application claims the benefit, under 35 U.S.C. §119(e) to U.S. provisional application Ser. No. 60/509,505, filed Oct. 8, 2003.

### STATEMENT REGARDING FEDERALLY-SPONSORED R&D

Not Applicable

### REFERENCE TO MICROFICHE APPENDIX

Not Applicable

### FIELD OF THE INVENTION

The present invention relates in part to transgenic animals which express a functional non-native B1 bradykinin receptor protein in place of a biologically active amount of the native form of the B1 bradykinin receptor. Transgenic animals of the present invention are generated by integration of a nucleotide sequence encoding a non-native form of a B1 bradykinin receptor protein, targeted directly into and replacing the region which controls expression of the native form of the receptor. Alternatively, the nucleotide sequence encoding a non-native form of a B1 bradykinin receptor protein is directed, either in a random or targeted fashion, onto a null B1 bradykinin receptor background. The transgenic animals of the present invention, including but not limited to the “humanized” mice exemplified herein, will be useful to evaluate the efficacy of human selective compounds in both in vitro and in vivo assays.

### BACKGROUND OF THE INVENTION

Dray and Perkins (1993, TINS 16: 99-104) and Proud and Kaplan (1988, Annual Review Immunology 6: 49-83) define two mammalian bradykinin receptor subtypes, B1 and B2, based on their pharmacological properties. The nonapeptide bradykinin (BK) and the decapeptide Lys-BK (kallidin) are liberated from the large protein precursor kininogen by the proteolytic action of kalikreins. BK and kallidin both activate the B2 receptor. These B2 receptor agonists are then degraded by a carboxypeptidase to produce the B1 receptor agonists des-Arg⁹BK and des-Arg¹⁰kallidin or by the angiotensin converting enzyme (ACE) to yield inactive peptides. BK and kallidin act as equipotent agonists at the B2 bradykinin receptor subtype. In contrast, BK is totally inactive at the B1 bradykinin receptor subtype.

The B2 and B1 bradykinin receptors are members of the superfamily of G-protein coupled receptors. Numerous mammalian B1 and B2 receptor genes have been isolated and characterized, including:

human B1 bradykinin—U.S. Pat. Nos. 5,712,111 and 5,965,367, both issued to Menke et al. on Jan. 28, 1998 and Oct. 12, 1999, respectively, as well as Menke et al. (1994, J. Biol. Chem. 269:21583-21586).

rabbit B1 bradykinin—MacNeil, et al., 1995, Biochem. Biophys. Acta 1264: 223-228.

mouse B1 bradykinin—Hess et al., 1996, Immunopharmacology 33: 1-8;

rat B2 bradykinin—McEachern, et al., 1991, Proc. Natl. Acad. Sci. 88, 7724-7728;

human B2 bradykinin—Hess, et al. (1992, Biochem. Biophys. Res. Comm. 184: 260-268); and,

rat B1 bradykinin—Jones, et al., 1999, Eur. J. Pharmacol. 374 (3), 423-433.

Hess et al. (1996, Immunopharmacology 33: 1-8) show that B1 receptor agonist selectivity is species specific, namely when comparing the mouse, human and rabbit B1 receptors.

Bock and Longmore (2000, Current Opin. in Chem. Biol. 4(4):401-407) present a recent update of known modulators of B1 and/or B2 bradykinin receptor activity. As reviewed by the authors, it is widely held in the scientific community that B2 receptors, but not B1 receptors, are expressed in normal tissue. In contrast, biologic processes which result in inflammation, pain, tissue damage can rapidly induce B1 receptor activity, as well as bacterial infection. The apparent inducibility of the B1 receptor under such pathological conditions may provide a therapeutic window for the use of B1 receptor antagonists as anti-inflammatory/analgesics, thus making the B1 receptor an attractive drug target.

WO 03/016495 discloses a transgenic rat which expresses human B1 bradykinin receptor along with the native form of the receptor.

Pesquero, et al. (2000, Proc. Natl. Acad. Sci., 97(14): 8140-8145) discloses B1 bradykinin receptor deficient mice. The authors show that these B1 receptor-deficient mice are healthy and fertile. Various phenotypes are disclosed, such as a blunting of bacterial LPS-induced hypotension, a reduced accumulation of polymorphonuclear leukocytes in inflamed tissue, as well as a reduction in spinal reflexes.

To this end, there remains a need for an animal model animal model that enables the analysis of compounds that are selective for a non-native form (e.g., human) of the B1 bradykinin receptor, relative to the rodent (e.g., rat or mouse) B1 bradykinin receptor, thus allowing for a model system to assess pharmacodynamic properties of potential modulators specificity to, say, the human B1 bradykinin receptor. The present invention meets this ongoing need by disclosing various “humanized” transgenic mouse models which express a human B1 bradykinin receptor protein on a null native B1 bradykinin receptor background.

### SUMMARY OF THE INVENTION

The present invention relates to non-human animal cells wherein at least one and preferably both alleles encoding the native form of the B1 bradykinin receptor has been rendered non-functional and in turn a functional transgene(s) encoding a non-native form of the B1 bradykinin receptor protein is integrated into the germ cells and/or somatic cells of the target animal. The generation of such transgenic non-human animal cells allows for further aspects of the present invention, namely: non-human transgenic embryos, non-human transgenic animals, including but not limited to initial founder animals, littermates, “floxed” animals, as well as animals where the floxed region has been excised (referred to herein as a “floxed-out” animal) and all subsequent offspring and progeny of these transgenic mice, wherein at least one and preferably both alleles encoding the native form of B1 bradykinin receptor has been rendered non-functional and have been replaced with a functional transgene encoding a non-native form of the B1 bradykinin receptor protein.

To this end, the present invention relates to non-human transgenic animal cells, non-human transgenic embryos, non-human transgenic animals and/or non-human transgenic founders, littermates, floxed, floxed-out animals as well as any and all subsequent offspring and progeny wherein at least one and preferably both alleles encoding the native form of B1 bradykinin receptor has been (1) rendered non-functional and (2) replaced with a functional transgene(s) operatively linked to a promoter fragment which wherein said transgene(s) encodes a human form of the B1 bradykinin receptor protein. The transgenic mice of the present invention provide for an animal model that enables the analysis of compounds that are selective for the human B1 bradykinin receptor, relative to the rodent (e.g., rat or mouse) B1 bradykinin receptor.

The present invention further relates to non-human transgenic animal cells, non-human transgenic embryos, non-human transgenic animals and/or non-human transgenic founders, littermates, floxed, floxed-out animals as well as any and all subsequent offspring and progeny wherein at least one and preferably both alleles encoding the native form of B1 bradykinin receptor has been (1) rendered non-functional and (2) replaced with a functional transgene(s) encoding a human form of the B1 bradykinin receptor protein wherein said transgene(s) are operatively linked to a promoter. In particular embodiments the non-native, humanized version of the B1 bradykinin receptor is operably linked to a promoter, such as the endogenous murine B1 bradykinin promoter.

Further embodiments of the present invention relate to cells and/or tissue derived from the transgenic animals disclosed herein, as well as cell lines generated by using such transgenic animals as a source to generate a cell line of interest. Therefore, an embodiment of the present invention relates to cells, tissues and/or cell lines derived from the transgenic mice of the present invention; transgenic mice which preferably carry a null phenotype for the native B1 bradykinin receptor protein and in turn comprise a transgene(s) encoding functional human B1 bradykinin receptor protein.

The present invention exemplifies a transgenic rodent, namely a transgenic mouse, in which a transgene encoding a functional form of a human B1 bradykinin receptor has been stably integrated into the germ cells and/or somatic cells of the target animal, preferably with the target animal ultimately showing a null phenotype for the native form of the B1 bradykinin receptor, allowing for production of chimeric mice and subsequent breeding to generate animal homozygous for the human B1 bradykinin receptor transgene. However, the present invention contemplates generation of other non-human transgenics, such as transgenic mice, transgenic rats, transgenic guinea pigs, transgenic rabbits, transgenic goats, transgenic non-human primates, such as chimpanzees, rhesus monkeys and African green monkeys, and transgenic cattle. Transgenic mice are preferred and exemplified herein.

As used herein, the term “functional” is used to describe a gene or protein that, when present in a cell or in vitro system, performs normally as if in a native or unaltered condition or environment. Therefore, a gene which is not functional (i.e., “non-functional”, “disrupted”, “altered”, or the like) will encode a protein which does not function as a wild type, native or non-altered protein, or encodes no protein at all. Such a non-functional gene may be the product of a homologous recombination event as described herein, where a non-functional gene is targeted specifically to the region of the target chromosome which contains a functional form of the gene, resulting in a “knock-out” of the wild type or native gene.

As used herein, a “modulator” is a compound that causes a change in the expression or activity of a mammalian B2 or B1 bradykinin receptor, such as a human B1 bradykinin receptor, or causes a change in the effect of the interaction of the respective receptor with its ligand(s), or other protein(s), such as an antagonist or agonist.

As used herein in reference to transgenic animals of this invention, we refer to “transgenes” and “genes.” A gene is a nucleotide sequence that encodes a protein, or structural RNA. The gene and/or transgene may also include genetic regulatory elements and/or structural elements known in the art. As used and exemplified herein, a transgene is a genetic construct including a gene. The transgene is integrated into one or more chromosomes in the cells in an animal by methods known in the art, such as exemplified herein by homologous recombination via a plasmid-based gene targeting construct. Once integrated, the transgene is carried at least one place in the genome, preferably a chromosome, of a transgenic animal. The transgene of interest is incorporated into the target genome of the mouse or other mammal, thus being introduced into their germ cells and/or somatic cells such that it is stably incorporated and is capable of carrying out a desired function. While a chromosome is the preferred target for stable incorporation of a transgene into the target animal, the term “genome” refers to the entire DNA complement of an organism, including nuclear DNA (chromosomal or extrachromosomal DNA) as well as mitochondrial DNA, which is localized within the cytoplasm of the cell. Thus, the transgenic mice of the present invention will stably incorporate one or more transgenes in either/or of the rodent's germ cells or somatic cells (preferably both), such that the expression of the transgene (e.g., a functional form of a human B1 bradykinin gene on a null native B1 background) achieves the desired effect of presenting an animal model to test the specificity of B1 receptor modulators against the human B1 receptor. It is preferable to introduce the transgene into a germ line cell, thereby conferring the ability to transfer the information to offspring. If offspring in fact possess some or all of the genetic information, then they, too, are transgenic animals.

As used herein, the term “animal” may include all mammals, except that when referring to transgenic animals, the use of this term excludes humans. It also includes an individual animal in all stages of development, including embryonic and fetal stages. A “transgenic animal” or more specifically, “non-human transgenic animal” is an non-human animal containing one or more cells bearing genetic information received, directly or indirectly, by deliberate genetic manipulation at a subcellular level, such as by microinjection, targeted gene delivery such as by homologous recombination, or infection with recombinant virus. As noted above, this introduced DNA molecule (e.g., transgene comprised within a gene targeting construct) can be integrated within a chromosome, or it can be extra-chromosomally replicating DNA. Contemplated animals include but are in no way limited to transgenic founders, littermates, floxed and floxed-out animals, any future subsequent offspring and progeny generated from these initial animals and of course any subsequent line (inbred or not) generated which harbors the genotypic results and/or phenotypic expectations of the intended gene targeting strategy.

As used herein, “rodent” relates to a species which is a member of the order Rodentia, having a single pair of upper and lower incisors for gnawing, wherein the teeth grow continuously and a gap is evident between the incisors and grinding molars. Preferred examples include for generation of transgenic animals include, but are not limited to transgenic mice (e.g., Mus musculus).

As used herein, “founder” refers to a transgenic animal which develops from the microinjected egg or blastocyst. The founders are tested for expression of a functional gene by any suitable assay of the gene product.

As used herein, a “floxed” animal is an animal which has received a gene(s), transgene(s) and/or targeting construct wherein a portion of that regions is flanked by sequence specific sites which allow for the controlled removal of the flanked sequence at a latter time. Floxed animals are exemplified herein by use of the known Cre recombinase system, which employs insertion of loxP sites to flank the sequence of interest (such as a selectable marker gene), with generation of Cre recombinase resulting in the removal of the flanked sequence within the genome of the transgenic animal.

As used herein, a “floxed-out” animal is an animal where the flanking sequences (such as an exemplified animal which has undergone Cre-mediated deletion (or ablation) of the pGK-neo cassette), and as described in the previous paragraph, have been excised.

As used herein, the term “line” refers to animals that are direct descendants of one founder and bearing at least one transgene locus stably integrated into their germline.

As used herein, the term “inbred line” refers to animals which are genetically identical at all endogenous loci. As used in the art, inbred lines may be used for including reproducibility from one animal to the next, ability to transfer cells or tissue among animals, and the ability to carry out defined genetic studies to identify the role of endogenous genes. Such inbred lines may be developed from such lines wherein the mice that are used for microinjection are members of established inbred strains.

As used herein, the term “genotype” is the genetic constitution of an organism.

As used herein, the term “phenotype” is a collection of morphological, physiological and/or biochemical traits possessed by a cell or organism that results from the interaction of the genotype and the environment. Included in this definition of phenotype is a biochemical trait wherein a non-native transgene has been introduced into the animal, thus altering its the genotypic profile, and whereby expression of this transgene(s) within the animal results in a new pharmacological selectivity to one or more chemical compounds, such a selectivity based on functional expression of the transgene(s) of interest. To this end, the term “phenotypic expression” relates to the expression of a transgene or transgenes which results in the production of a product, e.g., a polypeptide or protein, or alters the expression of the zygote's or the organism's natural phenotype.

The generation of a B1 bradykinin “humanized” mouse has not been reported and it was not evident that such a transgenic animal would mimic in vivo the compound selectivity of the human B1 bradykinin receptor. The essence of the present invention relates, to the demonstration that such a humanized transgenic animal possess such a phenotype, indicating the functional expression of this human receptor over the null phenotype of the native murine form, thus lending various assays described herein useful in selecting for modulators of the human B1 bradykinin receptor. The following examples are presented by the way of illustration and, because various other embodiments will be apparent to those in the art, the following is not to be construed as a limitation on the scope of the invention.

### BRIEF DESCRIPTION OF THE DRAWINGS

FIGS. 1A and 1B shows the targeting replacement vectors pB1R-KI1 (A) and pB1R-K2 (B), respectively, where the coding region of the human B1 bradykinin receptor replaces the coding region of the mouse B₁ bradykinin receptor. Mouse B1 bradykinin receptor 5′ and 3′ regions remain intact and the neo^{r} gene driven by a mouse pGK promoter is flanked by loxP sites for post-selection removal of this selectable marker. A thymidine kinase gene is placed outside the regions of 5′ and 3′ homology for use as a negative selection marker.

FIG. 2 shows the strategy for the direct replacement of the mouse B1 bradykinin receptor coding sequence with the human B₁ bradykinin receptor coding sequence. Directed homologous recombination into the targeted locus resulting in the human for mouse coding region replacement as well a the pGK-Neo cassette flanked by lox P sites. Treatment of cells or transgenic animals heterozygous or homozygous for this directed gene replacement may be floxed by administration of Cre recombinase or by breeding these transgenic animals with a Cre recombinase expressing mouse line.

FIGS. 3A and 3B show (A) a southern blot of an EcoRI digest of mouse genomic DNA probed with a 5′ external probe (as shown in FIG. 2) and (B) a PCR genotyping assay to confirm generation of replacement mice. Primer sets specific for both the wild type (native mouse) and human B1 receptor were developed. The mouse specific reaction yields a 319 bp fragment whereas the human specific fragment yields a 203 bp product.

FIGS. 4A and 4B show the effect of a human B1 receptor antagonist (Compound #1) on DAK-induced contraction in homozygous mouse (A) stomach fundus and (B) stomach ileum for experiment #1.

FIG. 5 shows the effect of a human B1 receptor antagonist (Compound #1) on DAK-induced contraction in homozygous mouse stomach fundus for experiment #2.

FIG. 6A-C show quantitative PCR was utilized to determine the levels of mouse B1 bradykinin receptor mRNA (open bar) and human B1 bradykinin receptor mRNA (striped bar) in brain, heart, normal ileum, induced ileum, induced fundus, lung, spinal cord, spleen and stomach of a (A) wild type, (B)-heterozygous and (C) homozygous transgenic animal.

### DETAILED DESCRIPTION OF THE INVENTION

The present invention relates to non-human animal cells wherein at least one and preferably both alleles encoding the native form of the B1 bradykinin receptor has been rendered non-functional and in turn a functional transgene(s) encoding a non-native form of the B1 bradykinin receptor protein is integrated into the germ cells and/or somatic cells of the target animal. The generation of such transgenic non-human animal cells allows for further aspects of the present invention, namely: non-human transgenic embryos, non-human transgenic animals, including but not limited to initial founder animals, littermates, “floxed” animals, as well as animals where the floxed region has been excised (referred to herein as a “floxed-out” animal) and all subsequent offspring and progeny of these transgenic mice, wherein at least one and preferably both alleles encoding the native form of B1 bradykinin receptor has been rendered non-functional and have been replaced with a functional transgene encoding a non-native form of the B1 bradykinin receptor protein.

To this end, the present invention relates to non-human transgenic animal cells, non-human transgenic embryos, non-human transgenic animals and/or non-human transgenic founders, littermates, floxed, floxed-out animals as well as any and all subsequent offspring and progeny wherein at least one and preferably both alleles encoding the native form of B1 bradykinin receptor has been (1) rendered non-functional and (2) replaced with a functional transgene(s) operatively linked to the endogenous mouse B1 bradykinin receptor promoter fragment which wherein said transgene(s) encodes a human form of the B1 bradykinin receptor protein. The transgenic mice of the present invention, provide for an animal model that enables the analysis of compounds that are selective for the human B1 bradykinin receptor, relative to the rodent (e.g., rat or mouse) B1 bradykinin receptor.

The present invention further relates to non-human transgenic animal cells, non-human transgenic embryos, non-human transgenic animals and/or non-human transgenic littermates of mouse origin, wherein at least one and preferably both alleles encoding the native mouse form of B1 bradykinin receptor has been (1) rendered non-functional and (2) replaced with a functional transgene(s) encoding a human form of the B1 bradykinin receptor protein. In particular embodiments the non-native, humanized version of the B1 bradykinin receptor is operably lined to a promoter, such as the endogenous murine B1 bradykinin promoter. As used herein, operably linked is used to denote a functional connection between two elements whose orientation relevant to one another can vary. In this particular case, it is understood in the art that a promoter can be operably linked to the coding sequence of a gene to direct the expression of the coding sequence while placed at various distances from the coding sequence in a genetic construct. The term “promoter” is well known in the art and as used herein is meant to denote a nucleic acid sequence or fragment involved in the binding of RNA polymerase to initiate gene transcription. A promoter as used herein may also optionally include additional cis-acting nucleic acid regions (e.g., enhancer sequences) that increase the utilization of a eukaryotic promoter, with an ability to function in either orientation and either upstream or downstream from the regions that initiates RNA transcription.

Further embodiments of the present invention relate to cells and/or tissue derived from the ansgenic animals disclosed herein, as well as cell lines generated by using such transgenic animals as a source to generate a cell line of interest. Therefore, an embodiment of the present invention relates to cells, tissues and/or cell lines derived from the transgenic mice of the present invention; transgenic mice which preferably carry a null phenotype for the native B1 bradykinin receptor protein and in turn comprise a transgene(s) encoding functional human B1 bradykinin receptor protein. Therefore, an embodiment of the present invention relates to transgenic mice which are non-functional for the native B1 bradykinin receptor protein and in turn comprises a transgene(s) encoding functional human B1 bradykinin receptor protein; as well as related transgenic mouse cells and embryos, which subsequently give rise to a transgenic mouse, including initial founder animals, littermates, “floxed” animals and all subsequent offspring and progeny mice which contain a null phenotype for native B1 bradykinin receptor while expressing a functional human B1 bradykinin receptor. The transgenic mice of the present invention provide for an animal model that enables the analysis of compounds that are selective for the human B1 bradykinin receptor, relative to the rodent (e.g., rat or mouse) B1 bradykinin receptor. In a specific embodiment of the present invention, such an animal model was generated by replacing the native mouse B1 bradykinin receptor coding region with the human B1 bradykinin receptors coding region via homologous recombination in embryonic stem cells. Embryonic stem cells with the correct homologous recombination event were used to develop mice which do not express functional amounts of mouse B1 bradykinin receptor protein while expressing the human B1 bradykinin receptor. It is shown herein that pharmacological and molecular analysis of homozygous B1 bradykinin receptor humanized mice exhibit properties associated with a human form of the receptor as opposed to a mouse form of the receptor.

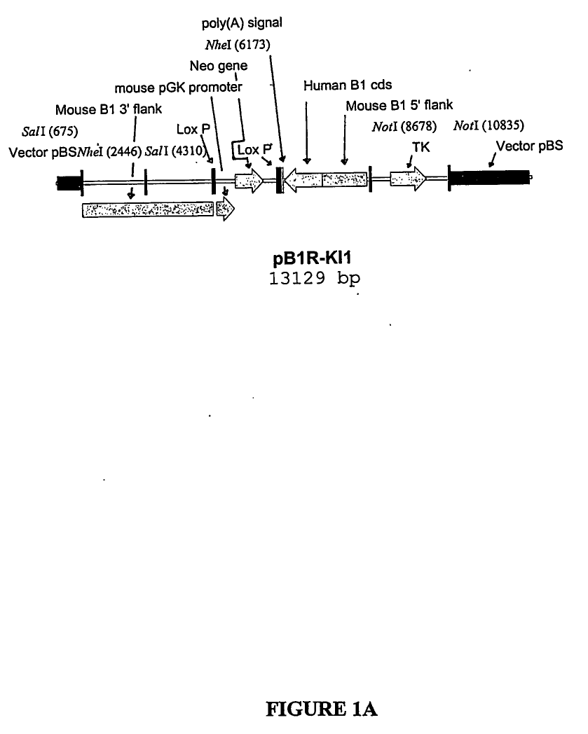

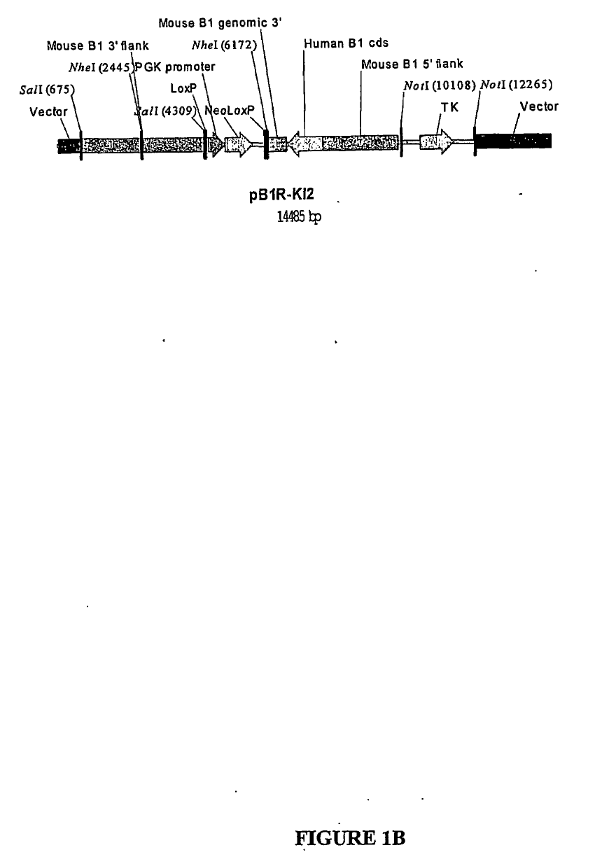

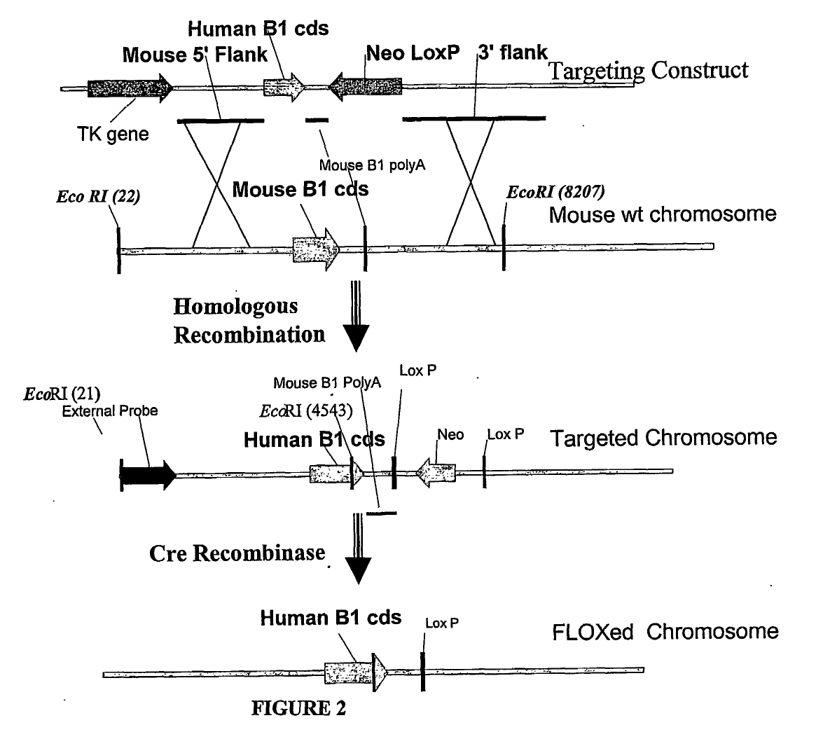

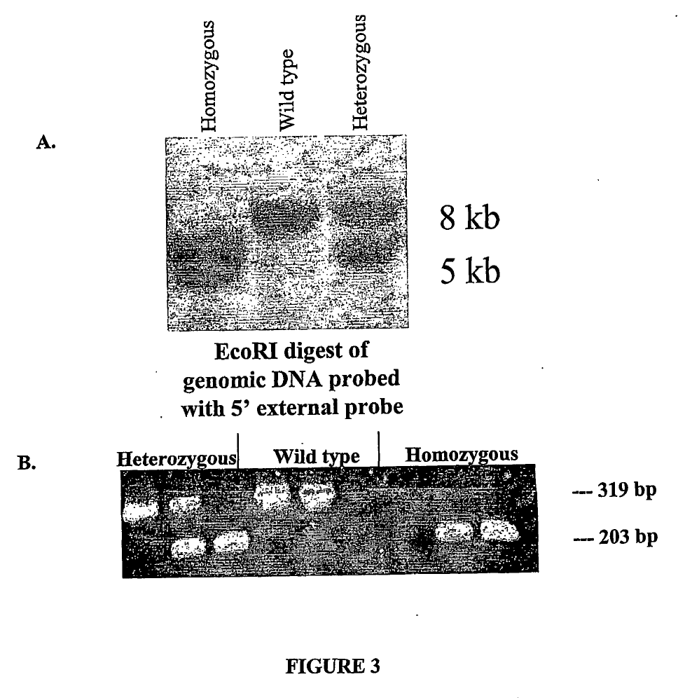

A specific embodiment of the present invention relates to non-human transgenic animal cells, non-human transgenic embryos, non-human transgenic animals and/or non-human transgenic littermates, wherein the human B1 bradykinin receptor coding sequence replaces the analogous murine B1 bradykinin receptor coding sequence by directed gene targeting through homologous recombination in embryonic stem (ES) cells. General principles regarding the construction of targeting constructs, directed homologous recombination in ES and selection methods are well known in that art and may be reviewed in numerous sources, for instance, in (1) Bradley et al., 1992, Bio/Technology 10: 534; and (2) Gene Targeting: A Practical Approach, 2^{nd} edition; Joyner, A. L. (ed) Oxford University Press, 2000; both of which are incorporated herein by reference. In an exemplification of the present invention, two targeting constructs are generated as shown in FIGS. 1A and 1B and a strategy for knocking in the human B1 bradykinin receptor coding sequence into the mouse B1 bradykinin receptor coding sequence region is described. Also noted in FIG. 2 is the ability to generate floxed-out animals whereby the coding regions expressing the positive selection marker has been removed by breeding the non-floxed animals (containing a pGK-neo cassette) to a mouse line which expresses Cre recombinase. Briefly, a targeting construct will comprise a human B1 bradykinin receptor coding sequence downstream of mouse B1 bradykinin receptor intronic sequences and either a human or mouse B1 bradykinin 3′ UTR region. Within the 3′ UTR region of the targeting construct resides a positive selection marker, such as a gene conferring resistance to neomycin (e.g., G418). This coding sequence is flanked by two LoxP sites, allowing for the removal of this positive selection marker at some point either prior to or subsequent to generation of the transgenic mice. This entire region is cloned within two extended regions of homology to the 5′ regulatory and 3′ UTR regions in order to promote a directed homologous recombination event whereby the human B1 bradykinin coding sequence replaces the mouse B1 bradykinin coding sequence within the mouse genome. Finally, the targeting vector may comprise a negative selection marker (such as the TK gene) which resides outside the region expected to integrated if the predicted homologous recombination event occurs. In this exemplified portion of the invention, 240 Neo^{r} ES clones were isolated for further analysis. Six “knock-in” clones from 80 of 240 clones were identified for further analysis and 2 knock-in ES clones were chosen for injection into blastocyts for implantation into female mice to generate chimeric mice which contain a germline-transmission of the human B1 bradykinin knock-in construct. Southern blot analysis (FIG. 3A) and PCR genotyping (FIG. 3B), as well as DNA sequence analysis, shows the expected pattern of allelic transmission from heterozygous crosses.

The exemplified knock-in mice disclosed herein exhibit pharmacological properties of the human, not the murine form of the receptor, as shown in Example section 6. This data shows a transgenic mouse homozygous for replacement of the human for mouse B1 bradykinin function, or biologically equivalent form thereof, shows a definable phenotype wherein the transgenic animal expresses an effective amount of the functional transgene product such that the transgenic animal now confers the selective pharmacological properties of the human B1 bradykinin receptor. This phenotype is detailed herein via a functional assay showing the effect of a selective antagonist of the human B₁ receptor. Two lines of human B1 bradykinin knock-in mice showed the predicted response to antagonize the B1 receptor in the presence of des-Arg¹⁰ kallidin (DAK). In wild type mice the DAK response of the stomach fundus is insensitive to the human B1 selective antagonist (referred to herein as “Compound #1”), as shown in Example section 6, whereas results with line 13 homozygous replacement mice indicate that the B1 response is blocked by Compound #1 (K_{b}=0.27 nM) and thus has properties of the human receptor. Therefore, the transgenic mice of the present invention, including but not limited to the humanized mice exemplified herein, will be useful to evaluate the efficacy of human selective compounds in both in vitro and in vivo assays. The addition of exogenous agonist, or the natural release of endogenous agonist resulting from a stimulus, may activate the human receptor. The ability of human selective compounds to antagonize the activation of the receptor will permit a determination of the efficacy of the compound in these animal models. To this end, the humanized B1 transgenic models as described herein will be useful to screen any potential modulator of receptor activity (e.g., antagonists or agonists), including but not necessarily limited to peptides, proteins, or non-proteinaceous organic or inorganic molecules. The transgenic animals of the present invention provide for improved models to evaluate the efficacy of human selective compounds in both in non-human animal models, such as rodents humanized for expression of the B1 receptor. The addition of exogenous agonist, or the natural release of endogenous agonist resulting from a stimulus, may activate the receptor. The ability of human selective compounds to antagonize the activation of the receptor will permit a determination of the efficacy of the compound. Such a transgenic animal overcomes historical problems associated with using wild type test animals to study the ability of small molecules to selectively (or not) modulate the human B1 receptor. Such assays revolved around treatment of test animal with an agent to increase wild type B1 bradykinin expression (such as bacterial lipopolysaccharides), which gave varying results to the extent in which B1 bradykinin expression was increased and which altered the properties of the blood-brain barrier. Even if successful, the properties of compounds selective for humans could not be assessed. To circumvent this problem, attempts were made to identify a species in which the pharmacological properties of the respective species matched the human B1 bradykinin receptor; a disadvantage being that one species may have similar-properties to the human with respect to one, but not all chemical series under consideration. Alternatively, species that are closely related genetically to human (such as non-human-primates) can be used. However, this alternative suffers from the low throughput in assaying compounds.

The humanized B1R mice may be evaluated for algesic response in response to mechanical, thermal, or chemical stimuli in animal models for inflammatory pain, such as treatment with complete Freund's adjuvant, carrageenan, formalin, streptozotocin, or lipopolysaccharide. In addition, naïve humanized B1R mice may be tested for responsiveness to thermal and chemical noxious stimuli, eg. Capsaicin. Finally the responsiveness to mechanical, thermal, or chemical stimuli in animal models for neuropathic pain, e.g. nerve ligation, can be assessed. If the phenotype of the B1R replacement mouse is similar to the wild type mouse and different from the B1R knockout mouse then the human B1R is expressed and responding to kinin system activation in the mouse. If the humanized B1R mouse responds appropriately to these stimuli then the ability of test compounds that selectively act on the human B1R can be tested. Human selective compounds will be active in the humanized mouse and not in the wild type mouse. Alternatively, if the humanized B1R mouse has a phenotype similar to the B1R knockout and dissimilar to the wild type mice this suggests the ability of the human B1R agonist, des-Arg¹⁰ kallidin and the mouse B1R agonist, des-Arg⁹bradykinin to elicit a response will be determined. If des-Arg¹⁰ kallidin, but not des-Arg⁹bradykinin, elicits a response then the human B1R expressed in the mouse has the potential to respond but the kinins generated in the rodent are ineffective in activating the human receptor. On the other hand, if neither des-Arg¹⁰ kallidin nor des-Arg9bradykinin are active in animals treated with agents that induce the mouse B1R then the induction of the human B1R may be impaired.

Analysis of the induction of the human B1R mice may be examined at the molecular level by quantitative PCR using RNA prepared from tissues. Comparison of these data with wild type and heterozygous mice will determine whether the human B1R is expressed in the appropriate tissues and levels. The induction of this receptor can be monitored in a like manner in animal models for inflammatory pain or neuropathic pain.

If the humanized B1R mouse responds to activation of the kinin system in an appropriate manner then it will serve for testing the efficacy of human selective on a variety of animal models. These include models of peripheral inflammatory processes such as vascular inflammation related to atherosclerosis, septic shock, airway inflammation, arthritis and angiogenesis in cancer. In addition the B1R may be involved in neuroinflammatory responses and thus the B1R mouse will be of utility in testing the efficacy of human selective B1R modulators in Alzheimer's disease models, brain edema, epilepsy and Parkinson's disease models. Thus this model has the potential to be of great value in evaluating the spectrum of therapeutic indications for which a compound that act on B1R may be effective.

A preferred embodiment of the present invention is the generation of transgenic rodents, especially transgenic mice, in which a transgene encoding a functional form of a human B1 bradykinin receptor has been stably integrated into the germ cells and/or somatic cells of the target animal, allowing for production of chimeric mice and subsequent breeding to generate animal homozygous for the human B1 bradykinin receptor transgene. However, the present invention contemplates generation of other non-human transgenics, such as transgenic mice, transgenic rats, transgenic guinea pigs, transgenic rabbits, transgenic goats, transgenic non-human primates, such as chimpanzees, rhesus monkeys and green african monkeys, and transgenic cattle. Transgenic mice are preferred and exemplified herein.

A preferred aspect in the generation of a non-human transgenic animal “humanized” for the B1 bradykinin receptor is either a directed replacement of the mouse gene, as exemplified herein, or possible insertion of the transgene of interest against a murine B1^{(-/-)} background, such as targeting ES cells isolated and cultured from such a bradykinin receptor murine B1^{(-/-)} background. Therefore, in some embodiments, the endogenous nonhuman B1 alleles are functionally disrupted so that expression of endogenously encoded murine B1 is suppressed or eliminated, so as to not interfere with expression of the human B1 transgene. A null background is generated by producing an animal with an altered native B1 bradykinin gene that is non-functional, i.e. a knockout. The animal can be heterozygous (i.e., having a different allelic representation of a gene on each of a pair of chromosomes of a diploid genome), homozygous (i.e., having the same representation of a gene on each of a pair of chromosomes of a diploid genome) for the altered B1 bradykinin gene, hemizygous (i.e., having a gene represented on only one of a pair of chromosomes of a diploid genome), or homozygous for the non-native B1 bradykinin gene, such as the exemplified humanized B1 bradykinin mice disclosed herein. Transgenes may be incorporated into embryonic, fetal or adult pluripotent stem cells (Capecchi, 1991, Science 244: 1288-1292, see also U.S. Pat. Nos. 5,464,764; 5,487,992; 5,627,059; 5,631,153 and 6,204,061, hereby incorporated by reference). The transgene can be introduced into the embryonic stem cells by a variety of methods known in the art, including electroporation, microinjection, and lipofection. Cells carrying the transgene can then be injected into blastocysts which are then implanted into pseudopregnant animals. In alternate embodiments, the transgene-targeted embryonic stem cells can be co-incubated with fertilized eggs or morulae followed by implantation into females. After gestation, the animals obtained are chimeric founder transgenic animals. The founder animals can be used in further embodiments to cross with wild-type animals to produce F1 animals heterozygous for the human B1 bradykinin receptor. In further embodiments, these heterozygous animals can be interbred to obtain the viable transgenic embryos whose somatic and germ cells are homozygous for the human B1 bradykinin receptor coding sequence. In other embodiments, the heterozygous animals can be used to produce cells lines. For example, a transgene may be contained within a gene targeting vector, wherein the vector contains homologous arms (see Cappecchi, supra) which can be used to direct a transgene to a specific genomic site within the target ES cell. Such foreign DNA can be incorporated into the embryonic stem cells by electroporation. Embryonic stem cells which carry the transgene in the appropriate fashion are injected into the inner cell mass of blastocysts. A chimeric animal is generated which is then crossbred to obtain animals wherein all cell carry the transgene. Along with microinjection described below, ES cell-based-techniques are a preferable methodology for generating transgenic mice. As exemplified herein, a common scheme to disrupt gene function by gene targeting in ES cells is to generate a targeting construct which is designed to undergo a homologous recombination with its chromosomal counterpart in the ES cell genome. The targeting constructs are typically arranged so that they insert additional sequences, such as a positive selection marker, into coding elements of the target gene, thereby functionally disrupting it. To this end, the present invention also relates to methods of producing nonhuman animals (e.g., non-primate mammals) that have the endogenous B1 gene inactivated by one gene targeting construct while directing, by homologous recombination, targeting the a second “humanized” targeting construct to a different region of the murine genome (e.g., a region promoting constitutive expression, such as the Rosa26 locus as described in U.S. Pat. No. 6,461,864, and hereby incorporated by reference.

As discussed throughout this specification, an aspect of this invention are transgenic animals having a transgene including a non-native gene on a native B1 bradykinin receptor null background. The method includes providing transgenic animals of this invention whose cells are heterozygous for a native gene encoding a functional B1 bradykinin receptor and an altered native B1 bradykinin receptor. These animals are crossed with transgenic animals of this invention that are hemizygous for a transgene including a non-native B1 bradykinin receptor gene to obtain animals that are both heterozygous for an altered native B1 bradykinin receptor gene and hemizygous for a non-native B1 bradykinin receptor gene. The latter animals are interbred to obtain animals that are homozygous or hemizygous for the non-native B1 bradykinin receptor and are homozygous or heterozygous for the altered native B1 bradykinin receptor gene. In particular embodiments, cell lines are produced and cells isolated from any of the animals produced in the steps of the method. Therefore, the native wild type gene is selectively inactivated in totipotent ES cells (such as those described herein) and used to generate the transgenic mice of the present invention. Techniques are available to inactivate or alter any genetic region to any mutation desired by using targeted homologous recombination to insert specific changes into chromosomal alleles (e.g., see Pesquero et al., 2000, Proc. Natl. Acad. Sci. 97: 8140-8145). Therefore, the present invention relates to diploid animal cells, non-human transgenic embryos, non-human transgenic animals and non-human transgenic littermates which are heterozygous or homozygous for a disrupted B1 bradykinin receptor gene resulting in deficient production of the B1 bradykinin receptor protein and which further serve as a background for addition of a non-native form of the bradykinin B1 receptor gene under the control of either an endogenous or heterologous promoter. The cells, embryos and non-human transgenic animals contain two chromosome alleles for B1 bradykinin receptor wherein at least one of the B1 bradykinin receptor alleles is mutated such that less than wild-type levels of B1 bradykinin receptor activity is produced. The diploid mouse cell, embryo or non-human transgenic mice homozygous for a disrupted B1 bradykinin receptor gene may show at least from about 50% to about 100% reduction in B1 bradykinin receptor activity compared to a wild type diploid cell. The diploid mouse cell, embryo or non-human transgenic mice heterozygous for a disrupted B1 bradykinin receptor gene may show at least from about 10% to about 100% reduction in B1 bradykinin receptor activity compared to a wild type diploid cell.

The nomenclature used herein and the laboratory procedures in transgenic protocols, cell culture, molecular genetics, and molecular biology are well known and commonly employed in the art. Standard techniques are used for recombinant nucleic acid methods, polynucleotide synthesis, cell culture, and transgene incorporation (e.g., electroporation, microinjection, lipofection). Generally enzymatic reactions, oligonucleotide synthesis, and purification steps are performed according to the manufacturer's specifications. The techniques and procedures are generally performed according to conventional methods in the art and various general references which are provided throughout this document. The procedures therein are believed to be well known in the art and are provided for the convenience of the reader. All the information contained therein is incorporated herein by reference.

The following examples are presented by the way of illustration and, because various other embodiments will be apparent to those in the art, the following is not to be construed as a limitation on the scope of the invention. For example, other methodology may be utilized to generate a “humanized” mouse of the present invention, including but not limited to including but not limited to (1) generation of a bradykinin B1 knock out to remove mouse BK1 function as a platform and then to “knock-in” a mammalian (e.g., human) BK1R in to the Rosa26 locus under the control of a general or tissue specific promoter; (2) generation of a bradykinin B1 knock out to remove mouse BK1 function as a platform and then use a BAC containing a specific mammalian BK1R (e.g., a human genomic BAC or a mouse BAC whereby the hBK1R genomic coding region was recombined onto the mouse BAC) to knock-in the respective BK1R gene, while retaining all required cis-acting regions of the BK1R promoter. Under this scenario, a preferred strategy would be to recombine the human gene recombined into a mouse BAC which is introduced into a B1 KO. Again, the artisan should consider the close proximity of the bradykinin B1 and B2 receptor genes and regulatory elements. BAC clones that contain the B1 promoter will also likely contain the B2 receptor gene as well. The artisan may also disrupt the B2 gene in the BAC clone provided there is a high degree of certainty that the region disrupted does not impact B1 receptor expression. If the B2 gene is not disrupted in the BAC transgene an alternative would be to introduce the humanized B1 mouse BAC into a B1/B2 double KO; (3) Generate a bradykinin B1 knock out to remove mouse BK1 function as a platform and introduction of a conventional transgenic mammalian (e.g., human) construct whereby a general or tissue specific regulatory construct was used to express the respective BK1R cDNA; and (4) using RNAi technology to knock-down endogenous mouse BK1R and then use the approaches in (1), (2) and/or (3) to introduce the transgene of interest. As noted above, methodology to generate such mice is known in the art and such art references are hereby incorporated by reference. In practicing one of these additional methodologies, it will be important for the artisan to understand the close proximity between the B2 receptor gene and the B1 promoter (10 kb or less between the end of the B2 receptor transcript and the start of the B1 receptor transgene). Therefore, it will be important to choose a targeting strategy that does not unknowingly compromise regulatory functions for either/or of the BK1R and BK2R promoter regions.

The generation of a B1 bradykinin “humaized” mouse has not been reported and it was not evident that such a transgenic animal would mimic in vivo the compound selectivity of the human B1 bradykinin receptor. The essence of the present invention relates to the demonstration that such a humanized transgenic animal possess such a phenotype, indicating the functional expression of this human receptor over the null phenotype of the native murine form, thus lending various assays described herein useful in selecting for modulators of the human B1 bradykinin receptor. The following examples are presented by the way of illustration and, because various other embodiments will be apparent to those in the art, the following is not to be construed as a limitation on the scope of the invention.

### EXAMPLE 1

### Construction of Human Bradykinin B1 Receptor Replacement Construct 1, pB1R-KI1

DNA sequence from GenBank Accession number AC068459 of chromosome 12 BAC clone RP23-158M23 was used in the design of constructs to replace the human B1 receptor with the mouse B1 receptor. The constructs are assembled by overlap extension PCR of individual pieces along with restriction endonucleases to facilitate the cloning of the DNA into plasmid vectors. The construct pB1R-KI1 is comprised of a 5′ mouse genomic DNA flanking region of 1.2 kb, the human B1 receptor coding sequence of 1059 bp along with the 126 nucleotides of 3′ untranslated genomic DNA that contains a poly(A) signal, a Neo cassette, for positive selection, that is flanked with Lox P sites, 7.4 kb of 3′ mouse B1 receptor flanking DNA, and a TK gene for negative selection. The steps taken in the preparation of this construct are described below. FIG. 1A shows the final targeting construct.

Step 1—Obtaining the mouse B1 genomic DNA 5′ flank: The 5′ flanking region of 1227 nucleotides was obtained by PCR with Pfu polymerase of mouse strain SV/J genomic DNA by first using the forward primer MmHsB1F3 5′-CCTCCTACTATCCCTAAGAGCG-3′ (SEQ ID NO:1) and the reverse primer Hybrid Primer R 5′-GGGCCAGGATGATGCCATCCACAGGAACCTGA AATTGAC-3′ (SEQ ID NO:2). The PCR conditions using the Pfu polymerase were 25 cycles of 94° C. for 25 sec, 60° C. for 25 sec and 72° C. for 2 min in a volume of 50 ul. For all the PCR steps in the generation of pB1R-KI1, an aliquot of approximately 1/10 of the PCR reaction was analyzed by agarose gel electrophoresis to determine the yield of the reaction, the purity of the product, and whether the product exhibited the expected size. The reaction products was purified and 0.25 ul of the primary reaction was used in a second semi-nested round of 25 cycles of PCR with the same cycling conditions as the primary reaction using the primer MnHsB1F1 5′-CCCT AAGAGCGAGTGAAAGG-3′ (SEQ ID NO:3) and the reverse primer Hybrid Primer R 5′-GGGCCAGGATGATG CCATCCACAGGAACCTGAAATTGAC-3′ (SEQ ID NO:2). The Hybrid Primer R contains 24 nucleotides that are identical to mouse genomic DNA sequence immediately upstream of the translation initiation codon for the mouse B1 receptor coding sequence and 18 nucleotides that are identical to the human B1 receptor coding sequence, including the translation initiation codon.

Step 2—Obtaining a portion of the human B1 receptor coding sequence for PCR overlap extension: A PCR fragment of 1083 nucleotides containing a portion of the human B1 receptor coding sequence by PCR with the forward primer Hybrid primer 5′GTCAATTTCAGGTTCCTGTGGATGGCATCATCCTGGCCC-3′ (SEQ ID NO:4) and the reverse primer MrHsB1R1 5′-AGGGAGCTGTTAGTGAAGGC-3′ (SEQ ID NO:5) was obtained by 2 rounds of PCR using the conditions described in the previous paragraph. Isolation and characterization of the human gene encoding the Bradykinin B1 receptor is disclosed in U.S. Pat. No. 5,712,111, issued Jan. 27, 1998, which is incorporated by reference herein. Hybrid primer F contains the 24 nucleotides that are identical to mouse genomic DNA sequence immediately upstream of the translation initiation codon for the mouse B1 receptor coding sequence and 18 nucleotides that are identical to the human B1 receptor sequence, beginning with the translation initiation codon.

Step 3—Connecting the products of step 1 and step 2 by PCR: The 1227 nucleotide 5′ flanking region from step 1 and the 1083 nucleotide human B1 receptor coding sequence PCR products from step 2 were purified with a Qiaquick column and eluted in 60 ul elution buffer. The purified products (1 ul each) were mixed and served as the template for a PCR reaction using the Pfu polymerase and the forward primer MmHsB1F1 5′-CCCTAAGAGCGAGTGAAAGG-3′ (SEQ ID NO:3) and the reverse primer MmHsB1R1 5′-AGGGAGCTGTTAGTGAAGGC-3′ (SEQ ID NO:5). The PCR conditions were 30 cycles of 94° C. for 25 sec, 60° C. for 25 sec and 72° C. for 3 min in a volume of 50 ul. The resulting product of approximately 2146 nucleotides was purified and subcloned into Topo Blunt PCR cloning vector. Several clones were selected and subjected to DNA sequence analysis to confirm the sequence of the product and identify any base changes that may have arisen during the PCR.

Step 4—Obtaining the human B1 receptor 3′ region containing the endogenous poly(A) signal: The human B1 receptor 3′untranslated region containing the endogenous poly(A) signal was obtained by PCR with human genomic DNA as template and the forward primer HuB1pAF 5′-GGCCGAAGGATAGAAAGACC-3′ (SEQ ID NO:6) and the reverse primer HSB1pAR_NotI 5′-CGGCGGCCGCTCATCAAGTCCAGGGA TTAGG-3′ (SEQ ID NO:7). The PCR conditions using the TaqGold polymerase were 40 cycles of 94° C. for 25 sec, 60° C. for 25 sec and 72° C. for 1 min in a volume of 50 ul. The resulting PCR fragment was subcloned into TOPO blunt PCR cloning vector and the DNA sequence of the clone was confirmed. To connect this product to the remainder of the human B1 receptor coding sequence a unique Eco RI site at position 818 of the human B1 receptor coding was exploited. The cloned-fragment was excised from Topo Blunt with the restriction endonucleases Eco RI and Not I and ligated with the human B1 receptor in the mammalian expression vector pcDNA3 digested with Eco RI and Not I. Clones of the resulting construct were confirmed by DNA sequence analysis.

Step 5—Connecting the mouse B1 5′flank/human B1 cds with human B1 3′ region containing poly(A) signal: Plasmid DNA from step 3, mouse B1 receptor 5′ flanking DNA connected to the human B1 receptor coding sequence, and step 4, the human B1 receptor coding sequence with the human B1 receptor 3′ untranslated region containing the endogenous polyA signal were combined in a PCR reaction. The forward primer MmHsB1F1 5′-CCCTAAGAGCGAGTGAAAGG-3′ (SEQ ID NO:3) and the reverse primer HSB1pAR-NheI 5′-GCGCTAGCTCATCAAGTCCAGGGATTAGG-3′ (SEQ ID NO:8) were used. The PCR conditions, using Pfu Turbo polymerase, were 31 cycles of 94° C. for 25 sec, 60° C. for 25 sec and 72° C. for 3 min in a volume of 50 ul.

Step 6—Ligating blunt×Nhe fragment from step 5 into Hpal×NheI pBSNeo TK—The product from step 5 of approximately 2.4 kb was purified and then digested with the restriction endonuclease Nhe I. This fragment was then ligated with Hpa I and Nhe I digested pBSNeoTK. The ligation was transformed into bacteria, plasmids were prepared and screened for the desired restriction pattern.

Step 7—Transferring the Not I by Sal fragment from pBSNeoTK derivative into pBluescript: A 4.4 kb fragment was isolated from the plasmid construct obtained in step 6 by digestion with restriction endonucleases Not I and Sal I. The 4.4 kb fragment was purified by agarose gel electrophoresis and subcloned into Not I/Sal I digested pBlueScript.

Step 8—Obtaining a mouse B1 receptor 3′ flanking genomic DNA: The 3′ flanking region of 7397 nucleotides was obtained by PCR from mouse genomic DNA from strain SV/J with the forward primer MR3f1_{—}2f 5′gcgtcgacTGGTTATTCCTAC AGCAACGG-3′ (SEQ ID NO:9) and the reverse primer MR3F1_{—}1r 5′gcgtcgacTGTGAG ATGCACACGTCAGC-3′ (SEQ ID NO:10). The resulting fragment was purified and subcloned into Topo Blunt PCR cloning vector and the DNA sequence of the insert was confirmed.

Step 9—Cloning mouse B1 genomic DNA 3′ flank into Sal I digested plasmid DNA from step 7: Clones with the 3′ flanking region insert from step 8 were digested with the restriction endonuclease Sal I and the 7.4 kb fragment was purified on an agarose gel. Plasmid DNA from step 7 was digested with Sal I, treated with shrimp alkaline phosphatase and then ligated with the 7.4 kb 3′ flank. The resulting clones were screened by restriction analysis for the correct orientation and subsequently confirmed by DNA sequence analysis.

Step 10—Addition of thymidine kinase (TK) gene: The plasmid construct prepared in step 6 was digested with restriction endonucleases Cla I and Dra. The digested DNA was treated with T4 DNA polymerase to form a blunt end at the ClaI site. A Not I linker was ligated to the blunt end and then the DNA was purified with a QiaQuick column. The purified DNA was digested with restriction endonuclease Not I and the fragments separated by agarose gel electrophoresis. A band of approximately 2153 nucleotides was excised and purified. The construct from step 9 was digested with Not I restriction endonuclease and treated with shrimp alkaline phosphatase, this was then ligated with the Not I digested fragment containing the TK gene. The ligation mix was transformed into XL-2 Blue chemically competent cells and clones were isolated by standard techniques. Clones were screened by restriction enzyme mapping, a clone with the correct pattern was selected and a large scale plasmid preparation was made to provide the DNA that was introduced into embryonic stem cells.

### EXAMPLE 2

### Construction of Human Bradykinin B1 Receptor Replacement Construct 2, pB1R-KI2

DNA sequence from GenBank Accession number AC068459 of chromosome 12 BAC clone RP23-158M23 was used in the design of pB1R-KI2, to replace the human B1 receptor with the mouse B1 receptor. The constructs are assembled by overlap extension PCR of individual pieces along with restriction endonucleases to facilitate the cloning of the DNA into plasmid vectors. The construct pB1R-KI2 is comprised of a 5′ mouse genomic DNA flanking region of 2.2 kb, the human B1 receptor coding sequence of 1059, 566 nucleotides of mouse genomic DNA that contains the endogenous B1 receptor poly(A) signal, a Neo cassette, for positive selection, that is flanked with Lox P sites, 3.6 kb of 3′ mouse B1 receptor flanking DNA, and a TK gene for negative selection. The steps taken in the preparation of this construct are described below. FIG. 1B shows the final targeting construct.

Step 1—Obtaining mouse genomic 5′ flanking DNA: PCR from mouse strain SvJ (˜50 ng) was done with the forward primers either MmGB1_F5 5′-TTAGCCATGGT TCAGTCACG-3′ (SEQ ID NO:11) or MGB1_{—}6F 5′-CACGACTCCTGTGAAT CAAGG-3′ (SEQ ID NO:12) in combination with the reverse primers MB1G_R 5′AAGGCTGTAGCTTCAGCGAG-3′ (SEQ ID NO:13) or MB1G_R2 5′TTTCT GCTATGGTTAGGCGC-3′ (SEQ ID NO:14). The PCR reaction was done with Expand polymerase for 35 cycles of 94° C. for 25 sec, 60° C. for 25 sec and 68° C. for 3 min in a volume of 50 ul. The product of either 2250 or 2457 nucleotides was purified. For all the PCR steps in the generation of pB1R-KI2, an aliquot of approximately 1/10 of the PCR reaction was analyzed by agarose gel electrophoresis to determine the yield of the reaction, the purity of the product, and whether the product exhibited the expected size

Step 2—Obtaining mouse B1 receptor 3′ flanking genomic DNA with endogenous poly(A) signal: The forward primer MuBAC.1f 5′-AAGGATGGTGGAGTTGAACG-3′ (SEQ D NO:15) and the reverse primer MuBAC.2r 5′-AAGCAGGTTGGATCCTACG-3′ (SEQ ID NO:16) were used to PCR a 3177 bp fragment of mouse strain SV/J genomic DNA. The PCR condition was done with Expand polymerase for 40 cycles of 94° C. for 25 sec, 60° C. for 25 sec and 68° C. for 9 min in a volume of 50 ul. The PCR product was purified.

Step 3—Connecting human B1 cds and mouse B1 receptor 3′ genomic DNA: The purified PCR product from step 2 was the template in a PCR reaction with MrpA_OF, 5′-CTTCCAACITCTGGCGGAATTAAGTGATGCACCTCTTTATAA-3′ (SEQ ID NO:17) in combination with the reverse primer MrpA_{—}1r 5′-gcgctagcCAAACTTGGCA AATCAGAGC-3′ (SEQ ID NO:18) to obtain a 598 nucleotide fragment. The forward MrpA_OF contains 25 nucleotides that are identical to the human B1 receptor coding, including the stop codon, and 22 nucleotides that are identical to mouse genomic DNA, including the stop codon for the mouse B1 receptor. The reverse primer MrpA_{—}1r contains 20 nucleotides that are identical to mouse genomic DNA and an Nhe I restriction endonuclease site. The human B1 receptor coding sequence was obtained by PCR using the forward primer Hybrid Primer F 5′-GTCAATTTCAGGTTCCTGTGGATGGCATC ATCCTGGCCC-3′ (SEQ ID NO:4) and the reverse primer MrpA_OR 5′-TTATAAAGAGGTGCATCACTTAATTCCGCCAGAAAAGTTGGAAG-3′ (SEQ ID NO:19) to obtain an 1102 nucleotide fragment. The forward primer Hybrid Primer F contains 24 bp that are identical to the mouse genomic DNA sequence, immediately upstream of the start of the coding sequence, followed by 18 bp identical to the human B1 receptor, including the translation initiation codon. The template for the PCR was a plasmid DNA containing the human B1 receptor coding sequence from the construction of pB1R-KI1 step 6. The PCR condition was done with Pfu polymerase for 20 cycles of 94° C. for 25 sec, 60° C. for 25 sec and 68° C. for 1 min 30 sec in a volume of 50 ul. The purified PCR products (3 ng each) were mixed in a second PCR reaction using the forward primer Hybrid Primer F 5′-GTCAATTTCAGGTTCCTGTGGATGGCAT CATCCTGGCCC-3′ (SEQ ID NO:4) and the reverse primer MrpA_{—}1r 5′-gcgctagcCAAACITGGCAAATCAGAGC-3′ (SEQ ID NO:20) to obtain a 1657 bp product. The PCR conditions were 20 cycles of 94° C. for 25 sec, 60° C. for 25 sec and 68° C. for 3 min in a volume of 50 ul with the Pfu polymerase.

Step 4—Connecting mouse B1 receptor 5′ genomic DNA with human B1 receptor coding sequence/mouse B1 receptor 3′ genomic DNA: Purified PCR product from step 1 was re-amplified with the forward primer MmGB1_{—}6F 5′-CACGACTCCTGTGA ATCAAGG-3′ (SEQ ID NO:21) and the reverse primer Hybrid Primer R 5′-GGGCCAGGATGATGCCATCCACAGGAACCTGAAATTGAC-3′ (SEQ ID NO:2). The product was purified and mixed with the final PCR product from step 3 (4 ng each). Overlap extension PCR with the forward primer MmGB1_{—}6F 5′-CACGACTCCTGTGAATCAAGG-3′ (SEQ ID NO:21) and the reverse primer MrpA-1r 5′-gcgctagcCAAACTTGGCAAATCAGAGC-3′ (SEQ ID NO:20) yielded a product of 3852 bp. The PCR conditions were 18 cycles of 94° C. for 25 sec, 60° C. for 25 sec and 68° C. for 4 min 30 sec in a volume of 50 ul with the Pfu polymerase.

Step 5—Cloning mouse B1 receptor 5′ genomic DNA/human B1 receptor coding sequence/mouse B1 receptor 3′ genomic DNA into pBSNeoTK: The final PCR product of 3852 nucleotides from step 4 was digested with the restriction endonuclease Nhe I. The vector pBSNeoTK was digested with restriction endonuclease Hpa I and Nhe I and ligated with the Nhe I digested PCR product (the other end of the PCR product is blunt as a consequence of using Pfu polymerase and therefore compatible with Hpa I). Clones of the resulting plasmid were screened for the correct orientation using restriction mapping, correct clones were analyzed by DNA sequence analysis to confirm the orientation and to identify any PCR induced mutations.

Step 6—Transferring the Not I by Sal I fragment from pBSNeoTK derivative into pBluescript: A 5.8 kb fragment was isolated from the plasmid construct from step 5 by digestion with restriction endonucleases Not I and Sal I. The 5.8 kb fragment was purified by agarose gel electrophoresis and subcloned into Not I/Sal I digested pBlueScript.

Step 7—Obtaining mouse B1 receptor 3′ flanking genomic DNA: PCR using Expand polymerase with mouse strain Sv/J genomic DNA was done with the forward primer mg3_F1 5′-ggtcgacTGGTTATTCCTACAGCAACGG-3′ (SEQ ID NO:22) and the reverse primer mg3_R2 5′-ggtcgacTGAACCAAGGCCACACTCTC-3′ (SEQ ID NO:23). The PCR yielded a fragment of 3628 nucleotides which was subcloned into TopoBlunt.

Step 8—Ligating mouse B1 receptor 3′ flanking DNA with plasmid DNA from step 6: The PCR product from step 7 was excised from TopoBlunt with the restriction endonuclease Sal I and purified by agarose gel electrophoresis. The plasmid produced in step 6 was digested with Sal I, treated with shrimp alkaline phosphatase, and ligated with the Sal I fragment containing the mouse B1 receptor 3′ flanking DNA. Clones were isolated and screened by restriction mapping for the correct orientation of the mouse B1 receptor flanking DNA. DNA sequence analysis of the clone was performed to confirm the construct and to identify any PCR induced errors.

Step 9—Adding the thymidine kinase (TK) gene: The plasmid construct prepared in step 6 of example 1 was digested with restriction endonucleases Cla I and Dra. The digested DNA was treated with T4 DNA polymerase to form a blunt end at the ClaI site. A Not I linker was ligated to the blunt end and then the DNA was purified with a QiaQuick column. The purified DNA was digested with restriction endonuclease Not I and the fragments separated by agarose gel electrophoresis. A band of approximately 2153 nucleotides was excised and purified. The construct from step 8 was digested with Not I restriction endonuclease and treated with shrimp alkaline phosphatase, this was then ligated with the Not I digested fragment containing the TK gene. The ligation mix was transformed into XL-2 Blue chemically competent cells and clones were isolated by standard techniques. Clones were screened by restriction mapping, a clone with the correct pattern was selected and a large scale plasmid preparation was made to provide DNA that was introduced into embryonic stem cells.

### EXAMPLE 3

### Generation of Human Bradykinin B1 Receptor Knock-In (KI) Mice

Materials and Methods—ES cell transfection: V6.5 mouse ES cells (Eggan, et al., 2001, Proc. Natl. Acad. Sci. USA 98: 6209-6214) were cultured as described (Joyner, A. L. (Ed.), 2000, Gene Targeting: A Practical Approach (Oxford University Press, New York) in ES cell qualified DMEM with 15% FBS containing 1,000 units/ml ESGRO on mitomycin C treated neomycin-resistant primary feeder fibroblasts (all ES cell culture reagents were from Cell & Molecular Technologies, New Jersey, USA). Electroporation was carried out using 20 μg of Not I-linearized pB1R-KI2 targeting vector and approximately 6×10⁶ ES cells as described (Joyner, A. L. (Ed.), 2000, Gene Targeting: A Practical Approach (Oxford University Press, New York). Selection for neomycin resistant and thymidine kinase (TK) negative colonies was carried out 30 h later and subsequently for 7 days with 250 μg/ml Geneticin (Invitrogen, California, USA) and 2 μM ganciclovir (Novagen, Wis., USA) (see FIG. 2 for schematic review of the targeting process). ES colonies were picked and genomic DNA was isolated as described (Laird, et al., 1991, Nucleic Acids Res. 19: 4293). Targeted ES clones were identified by Southern-blot analysis of genomic DNA digested with EcoR I, using a 1.1 kb 5′ external probe which was prepared by polymerase chain reaction (PCR) from mouse genomic DNA using two primers (5′ primer: 5′-CTCAACAAACCCTGGACATC-3′ [SEQ ID NO:24]; and 3′ primer: 5′-GAAGTCTGAGGAGA GAAGTG-3′ [SEQ ID NO:25] and confirmed by direct DNA sequencing. The wild-type and targeted alleles were identified as a 8.2 kb and a 4.5 kb fragment, respectively. Of 80 ES clones analyzed, six were identified and characterized as targeted carrying a single homologously recombined knock-in allele.

Microinjection and animal breeding—All animal protocols used herein were approved by the Merck Research Laboratories Animal Care and Use Committee in West Point, Pa. ES cells were injected into C57BL/6 (Taconic Farms, New York, USA) host blastocysts to generate chimeras as described (Joyner, A. L. (Ed.), 2000, Gene Targeting: A Practical Approach (Oxford University Press, New York), which were subsequently crossed with C57BL/6 mice to derive mice bearing a germline-transmitted B1 knock-in allele. Genomic DNA was isolated as described (Laird, et al., id.) and genotyping was carried out by Southern-blot analysis as described above (see FIG. 3A). Mice heterozygous for the knock-in allele were inter-crossed to generate homozygous knock-in progenies.

Results—Generations of Mice Carrying a Human B1 Receptor Knock-in Allele—

Two independent targeted ES clones were microinjected and chimera derived from both ES clones transmitted the knock-in allele through germline. Mice heterozygous for the knock-in allele were grossly normal and transmitted the allele at expected ratios. Mice homozygous for the knock-in allele also show normal growth and behavior.

### EXAMPLE 4

### PCR Genotyping Assay for Human B1 Replacement Mice

A primer set specific for the wild type mouse B1 receptor and a second set specific for the human B1 receptor were developed. The mouse specific reaction using the forward primer MmB1f 5′-TTCTAACCAAAGCCAGCAGG-3′ (SEQ ID NO:26) and the reverse primer MmB1r 5′-GGCAGAGGTCACTTCCAAAG-3′ (SEQ ID NO:27) yields a 319 bp product that is specific for the mouse B1 receptor chromosome. The human specific reaction using the forward primer HsB1f 5′-TCTTATTCCAGGTGCAAGCAG-3′ (SEQ ID NO:28) and the reverse primer HsB1r 5′-GGGATGAAGATATTGGAGCAAGAC-3′ (SEQ ID NO:29) yields a product of 203 bp that is specific for the chromosome in which the human B1 receptor coding sequence is present (FIG. 3B). The PCR conditions in a volume of 20 ul using the TaqGold polymerase were, 10 min 94° C. then 35 cycles of 94° C. for 30 sec, 60° C. 30 sec, and 72° C. for 1 min. The primer concentration is 10 pmoles per reaction, the mouse genomic DNA template is approximately 50 ng/reaction and the MgCl₂ is 1.5 mM final concentration. The genotyping assay can be used either in a single or multiplex reaction, reaction products are analyzed on a 2% NuSieve agarose gel.

### EXAMPLE 5

### DNA Sequence Analysis of Human B1 Replacement Chromosome

The integrity of the human B1 receptor in replacement 2 line 12 and line 13 mice was confirmed by DNA sequence analysis. The forward primer Mf_{—}1f 5′-CTGGTCTGCCATCATAACG-3′ (SEQ ID NO:30) and the reverse primer Mf_{—}2r 5′-CAGGATCAGCCTAATCTCCG-3′ (SEQ ID NO:31) were based on mouse genomic DNA sequences flanking the human B1 receptor coding sequence and generate a product of 1481 nucleotides. The fragment was amplified from approximately 100 ng of mouse genomic DNA using Expand polymerase in a 50 ul reaction volume. Following a 1 min preincubation at 94° C., there were 35 cycles of 94° C. for 30 sec, 60° C. for 30 sec and 70° C. for 1 min 30 sec. The PCR products were evaluated on an agarose gel and then purified using a QiaQuick column. The PCR products were sequenced by standard cycle sequencing protocols and analyzed on an ABI 377. As expected products from homozygous replacement mice contained a portion of flanking mouse genomic DNA and the complete human B1 receptor coding sequence. In addition, at the mouse/human junction the DNA sequence of heterozygous mice was mixed. These data confirm the gene targeting and the integrity of human B1 receptor coding sequence.

### EXAMPLE 6

### Functional Assay

### B1 Receptor Activity in Homozygous Human B1 KO Mice

A functional assay was carried out twice on homozygous human b1 mouse fundus and ileum tissue to evaluate its B1 receptor activity in line 13, a mouse line homozygous for replacement of the mouse B1 gene with the human B1 receptor gene. A selective human B1 receptor compound (Compound #1) was tested for antagonistic activity against the receptor from these knock-in mice. Specifically, two homozygous mice were euthanized with CO₂. Their stomach and ileum were removed and the fundic portion was divided in half by cutting an incision along the greater curvature. A medial longitudinal strip about 1 mm×0.6 cm was obtained by making cut along the midline incision. Two fundic strips were obtained from each stomach. The ileum was cut about 4 cm from the cecum. Each ileum is divided into two pieces and whole muscle was used. Tissues were placed in a 5 ml jacked holders containing Krebs buffer (118 mM: NaCl, 4.7 mM KCl, 2.5 mM CaCl₂, 1.2 mM MgSO4, 1.2 mM KH₂PO₄, 25 mM NaHCO₃ and 11.1 mM dextrose) and bubbled with 5% CO₂/95% O₂ at 37° C. Each piece of ileum was tied one end to a glass rod with silk 4-0 and the other end to the Statham—Gould force transducer. One gram tension was applied to each tissue and contraction was recorded by a Hewlett-Packard 7758 recorder. All tissues receive 50 mM KCl at the end of 1 hr equilibrium for maximal response. The tissues are washed every 20 min for one hour. 30 nM of Compound #1 was added to two treated ileum or two treated fundus tissues and incubated for one hour. 0.1 μM M422 and 1 μM thiorphan are added to the bath 30 min before DAK dosing. A cumulative dose-response of DAK (10-¹⁰M to 3×10⁻⁵M) is carried out after pre-incubation.

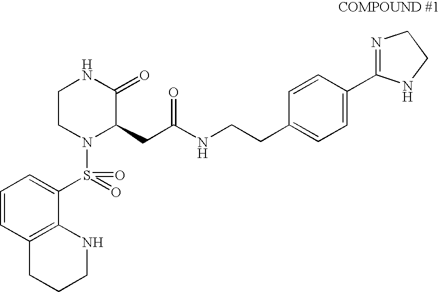

Table 1 shows the sample dilution for DAK and Compound #1. DAK is diluted in Krebs solution. Full log dilution is made by adding 25 μl in 250 μl. Compound #1 is diluted in DMSO.

TABLE 1

| | Compound | dilution range | Stock | dilution | |
|---|---|---|---|---|---|
| 1. | Compound | 3 × 10⁻⁸ | 10 | 300x | 3 μl in 300 μl, then |
| | #1 | | mM | | 60 μl in 200 μl |
| 2. | DAK | 10⁻¹⁰-3 × 10⁻⁵ | 5 | 5x | 50 μl in 250 μl, then |
| | | | mM | | 25 μl in 250 μl |

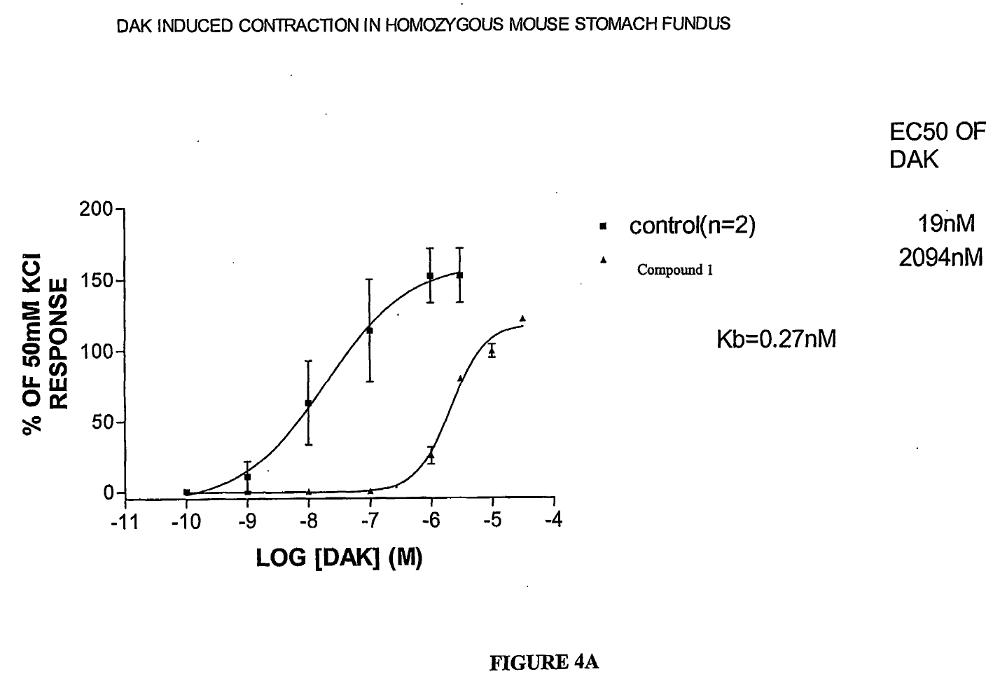

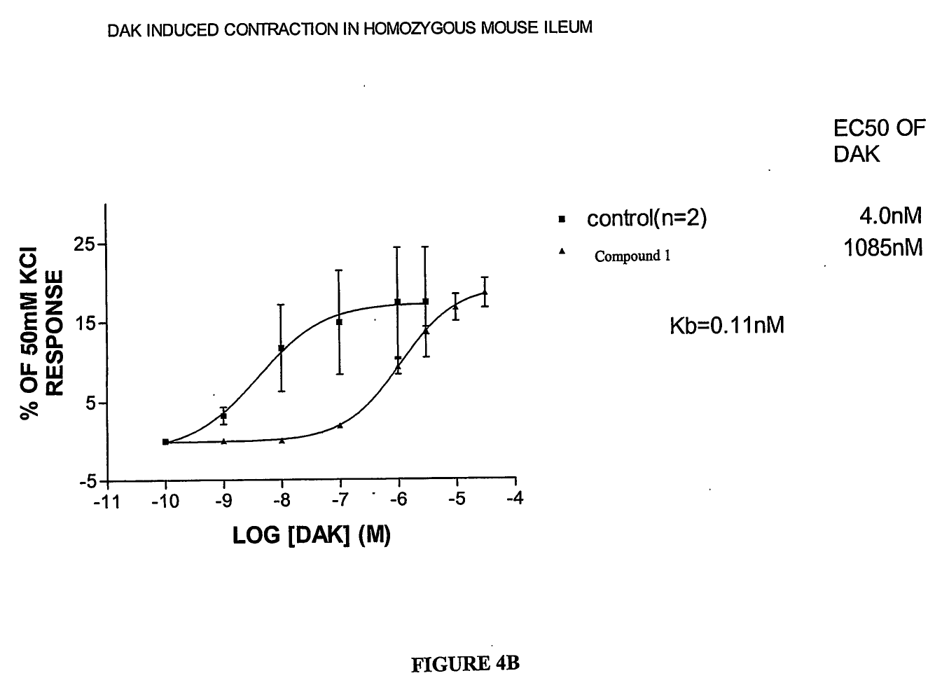

Results—(Experiment #1): Line #13 homozygous mice demonstrated full DAK contractile responses as in the wild type. However, these mice showed human bradykinin B1 receptor activity instead of rat B1 activity. The Kb for Compound #1 was 0.27 nM in fundus and 0.11 nM in ileum. These data are in accordance with the binding data (hb1=0.04 nM, rat b1=591 nM). Representative data is shown below in Table 2 as well as in FIGS. 4A and 4B.

TABLE 2
1) homozygous mouse fundus
EC₅₀ of des-arg¹⁰-kallidin

| | | | |
|---|---|---|---|
| Control | (n = 2) | 19 nM | |
| 30 nM L-878365 | (n = 2) | 2094 nM | Kb = 0.27 nM |

2) homoxygous mouse ileum
EC₅₀ of des-arg¹⁰-kallidin

| | | | |
|---|---|---|---|
| Control | (n = 2) | 4.0 nM | |
| 30 nM L-878365 | (n = 2) | 1085 nM | Kb = 0.11 nM |

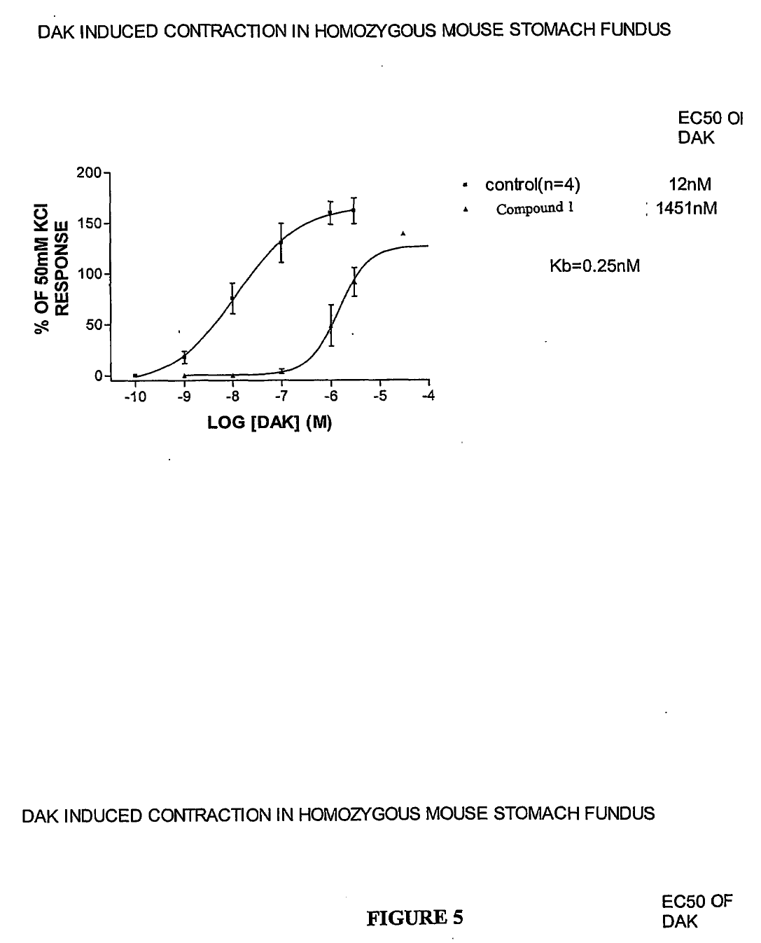

Results—(Experinent #2): Line #13 homozygous mice demonstrated full DAK contractile responses as in the wild type. As in Experiment #1, these B1 receptor humanized mice showed human B1 instead of rat B1 activity. The Kb for Compound #1 was 0.23 nM in fundus and 0.21 nM in ileum. These data are in accordance with the binding data (hb1=0.04 nM, rat b1=591 nM). Representative data is shown below in Table 3 as well as in FIG. 5.

TABLE 3
1) homozygous mouse fundus
EC₅₀ of des-arg¹⁰-kallidin

| | | | |
|---|---|---|---|
| Control | (n = 2) | 8.8 nM | |
| 30 nM Compound #1 | (n = 2) | 1142 nM | Kb = 0.23 nM |

Combined with Experiment #1

| | | | |
|---|---|---|---|
| Control | (n = 4) | 12 nM | |
| 30 nM Compound #1 | (n = 4) | 1451 nM | Kb = 0.25 nM |

2) homozygous mouse ileum
EC₅₀ of des-arg¹⁰-kallidin

| | | | |
|---|---|---|---|
| Control | (n = 2) | 0.83 nM | |
| 30 nM Compound #1 | (n = 2) | 118 nM | Kb = 0.21 nM |

Combined with Experiment #1

| | | | |
|---|---|---|---|
| Control | (n = 4) | 1.7 nM | |
| 30 nM Compound #1 | (n = 4) | 5941 nM | Kb = 0.09 nM |

### EXAMPLE 7

### Removal of pGKneo by Cre-Mediated Recombination

Mice which were either heterozygous or homozygous for the human B1R receptor were bred to Rosa-SA-Cre mice. Cre positive progeny that were heterozygous for the human B1R receptor were interbred. Genomic DNA was prepared from the progeny of the humanB1R+/−, Cre+ intercross and tested for the presence of the pGKneo gene linked to the human B1R gene by PCR. To test for the presence of the Neo gene a forward primer Neo2f, 5′-CTGACCGCTTCCTCCT GACCGCITCCTCGTGCTTTAC-3′ (SEQ ID NO:32) located within the neo gene was used in combination with a reverse primer Neo2r, 5′-CGGAGGACA GAGTAATCGG-3′ (SEQ ID NO:33) located in mouse genomic DNA. The PCR conditions, using TaqGold polymerase, were 10 min 95° C. followed by 35 cycles of 95° C. for 30 sec, 60° C. for 30 sec and 72° C. for 1 min with the template being 50 ng of mouse genomic DNA. The amplification of a product of 937 nucleotides is diagnostic of the presence of the Neo gene.

PCR with a second primer set was diagnostic of the Cre mediated deletion of the pGKneo cassette. The forward primer Flox1f 5′-GAGGACAGAGTAATCGG-3′ (SEQ ID NO:34) and the reverse primer Flox1r 5′-GCTCCCTGTTCCGAGTTAGG GCTCCCTGTTCCGAGTTAGG-3′ (SEQ ID NO:35) were used in a PCR reaction using 50 ng of mouse genomic DNA and the following conditions with TaqGold polymerase; 10 min 95° C. followed by 35 cycles of 95° C. for 30 sec, 60° C. for 30 sec and 72° C. for 1 min. The Flox1f×Flox1r primer set amplifies a 579 nucleotide product from the wild type mouse chromosome and a 688 nucleotide product from the chromosome which has undergone Cre mediated deletion of the pGKneo gene. Amplification of the chromosome containing pGKneo results in a 2447 nucleotide product. With the PCR conditions used in the assay this product is not amplified efficiently. The 109 nucleotide difference between the wild type mouse chromosome and the chromosome containing the human B1 receptor coding sequence that has undergone Cre mediated deletion of pGKneo arises from the presence of the 34 nucleotide LoxP site and sequences that lie between the cloning site used to introduce mouse genomic DNA into the original construct and the LoxP sites. This difference serves as an efficient diagnostic pattern for homozygous wild type chromosome, single band of 579 nucleotides, heterozygous mice two bands of 579 and 688 nucleotides and homozygous humanized B1R replacement mice a single band of 688 nucleotides.

### EXAMPLE 8

### Determination of Bradykinin B1 Receptor mRNA Levels

The quantity of bradykinin B1 receptor mRNA was determined by real-time PCR using TaqMan technology (Applied Biosystems, Foster City Calif.). A primer set and probe that is specific for detecting the mouse B1 receptor mRNA was developed. The mouse specific forward primer has the sequence 5′-AGCTGTGAG CTCTTGTTTTCTGT-3′ (SEQ ID NO:36) and the reverse primer has the sequence 5-TTTGGTTAGAAGGCTGTAGCTTCA-3′ (SEQ ID NO:37), this primer set amplifies a product of 82 base pairs that spans an intron/exon boundary. The mouse specific product is detected with the probe 6FAM-GGACGCCATCCACAGGA ACCCA-TAMRA (SEQ ID NO:38). The primer/probe set that is specific for the human B1 receptor mRNA detects a transcript that is a hybrid between the mouse 5′UTR in one exon and the human B1 receptor coding sequence present on a second exon. This chimeric transcript, which is diagnostic of the presence of human bradykinin B1 receptor nucleotide sequence, is detected with the forward primer 5′-GCTGAAGCTGTGAGCTCITTGTT-3′ (SEQ ID NO:39) the reverse primer 5′-GGTTGGAGGATTGGAGCTCTAGA-3′ (SEQ ID NO:40) which amplifies a product of 82 base pairs that spans an intron/exon boundary. The mouse/human product is detected with the fluorescent probe 6FAM-TGCCATCCACAGGAACCCAGACAG-TAMRA (SEQ ID NO:41).

Tissues were removed from three mice of each genotype, either wild type for the mouse B1 receptor DNA sequence, heterozygous, or homozygous for human B1 receptor DNA sequence, as determined by genotype analysis of mouse genomic DNA as described in Example 4. The tissues sampled included, brain, spinal cord, ileum, stomach, heart, lung and spleen. In addition, samples from the ileum and fundus were obtained from tissue that had been incubated in a manner resulting in the induction of the B1 receptor mRNA as described in Example 6 for testing B1 receptor function. These samples were termed induced fundus or induced ileum The tissues were homogenized in Qiagen Buffer RLT using a polytron. Total RNA was prepared using the Qiagen RNeasy midi prep kit and following the protocol described by the manufacturer (USA-Qiagen, Valencia Calif.). The total RNA was further purified to mRNA using the Qiagen Oligotex procedure. The mRNA samples were analyzed on an Agilent 2100 Bioanalyzer (Agilent Technologies Inc., Palo Alto, Calif.) to determine purity and quantity. The mRNA was used to prepare cDNA with the Invitrogen Superscript Choice cDNA Synthesis System (Invitrogen Corp., Carlsbad, Calif.) according to the manufacturer's instructions. The cDNA was purified and concentrated using a Qiaquick spin column (USA-Qiagen, Valencia Calif.) and then diluted to 1 ng/ul prior to TaqMan analysis.

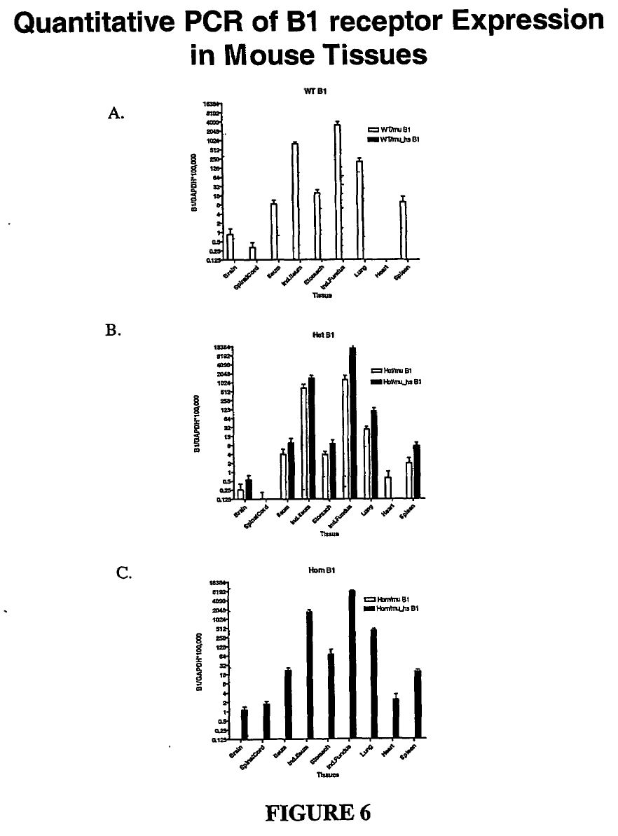

TaqMan reactions consisted of 5 ul of 1 ng/ul cDNA and 5 ul of a primer probe mix at a final concentration of 200 nM. The primer/probe mix consisted of either the mouse specific forward and reverse primers along with the mouse specific probe or the mouse/human hybrid specific forward and reverse primers along with the human specific probe. A final concentration of 200 nM of a GAPDH primer/probe set (Applied Biosystems, Foster City, Calif.) was included in the primer/probe mix as an internal control for all reactions. The total reaction volume of 20 ul was achieved with 10 ul of 2× Universal PCR Master Mix (Applied Biosystems, Foster City, Calif.). The reactions were run on either an Applied Biosystems PRISM 7000 Sequence Detection System, in a 96-well format, or an Applied Biosystems PRISM 7900HT Sequence Detection System, in 384-well format. Each sample was tested in triplicate. Five-point standard curves for each primer/probe set were performed as part of each experiment by doing a 10-fold serial dilution starting at 100,000 copies per well each of plasmid DNA containing rodent GAPDH and plasmid DNA containing either the mouse B1 receptor or the chimeric mouse/human B1 receptor Data was analyzed by first normalizing to the standard curve for the appropriate transcript and then to the amount of GAPDH transcript detected in each well. The results of the three individual animals for each genotype were combined and the mean and standard error graphed as shown in FIG. 6A-C. The results indicate that there the pattern and the level of expression of the mouse/human B1 receptor transcript is similar to that of the endogenous mouse B1 receptor. Furthermore it is evident that the mouse/human hybrid transcript can be induced in a manner analogous to the endogenous mouse B1 receptor.

What is claimed is:

1. A non-human transgenic animal having a genome comprising at least one copy of a transgene encoding a human bradykinin B1 receptor protein, or a functional equivalent thereof, and which exhibits a null phenotype for the native form of the bradykinin B1 receptor, such that the transgenic animal demonstrates a humanized B1 bradykinin receptor binding profile.

2. A non-human transgenic animal of claim 1 wherein the non-human transgenic animal is a mouse.

3. A non-human transgenic animal of claim 1 wherein the transgene encoding human bradykinin B1 receptor protein, or portion thereof, directly replaces the gene encoding the native for of bradykinin B1.

4. A non-human transgenic animal of claim 3 wherein the non-human transgenic animal is a mouse.

5. A non-human transgenic animal of claim 3 wherein the transgene encoding human bradykinin B1 receptor protein, or portion thereof, directly replaces the gene encoding the native for of bradykinin B1 and said human B1 bradykinin gene is operatively fused to the native bradykinin B1 receptor protein.

6. A non-human transgenic animal of claim 5 wherein the non-human transgenic animal is a mouse.

7. A non-human transgenic animal which comprises a gene targeting construct containing at least a nucleotide sequence encoding a human bradykinin B1 receptor protein, or a functional equivalent thereof, and a floxed marker gene, and which exhibits a null phenotype for the native form of the bradykinin B1 receptor, such that the transgenic animal demonstrates a humanized B1 bradykinin receptor binding profile.

8. A non-human transgenic animal of claim 7 wherein the non-human transgenic animal is a mouse.

9. A non-human transgenic animal of claim 7 wherein the gene targeting construct has been inserted into the genome of the transgenic animal so as to directly replace the gene encoding the native for of bradykinin B1.

10. A non-human transgenic animal of claim 9 wherein the non-human transgenic animal is a mouse.

11. A non-human transgenic animal of claim 8 wherein the floxed gene has been excised.

12. A non-human transgenic animal of claim 10 wherein the floxed gene has been excised.
